(19) United States
(12) Patent Application Publication
(10) Pub. No.: US 2020/0129043 A1
(43) Pub. Date: Apr. 30, 2020

(54) TUBULAR INSERTION DEVICE AND OPERATION SUPPORT METHOD

(71) Applicant: OLYMPUS CORPORATION, Tokyo (JP)

(72) Inventors: Koichi TAKAYAMA, Kokubunji-shi (JP); Hiromasa FUJITA, Hachioij-shi (JP)

(73) Assignee: OLYMPUS CORPORATION, Tokyo (JP)

(21) Appl. No.: 16/732,456

(22) Filed: Jan. 2, 2020

Related U.S. Application Data
(63) Continuation of application No. PCT/JP2017/024833, filed on Jul. 6, 2017.

(51) Int. Cl. A61B 1/005; A61B 1/00

(52) U.S. Cl. CPC A61B 1/005; A61B 1/00043

(57) ABSTRACT

A tubular insertion device includes a tubular device including a flexible tube portion to be inserted into a subject, a sensor which detects a disposition state of the flexible tube portion in the subject, a prediction calculator which calculates next operation information that is information regarding an operation to be performed next by the tubular device, based on the disposition state detected by the sensor and stored data regarding an operation of the tubular device corresponding to each disposition state of the flexible tube portion, and an output circuit which outputs the next operation information calculated by the prediction calculator.

### CROSS-REFERENCE TO RELATED APPLICATION

This is a Continuation Application of PCT Application No. PCT/JP2017/024833, filed Jul. 6, 2017, the entire contents of which are incorporated herein by reference.

### BACKGROUND

The present invention relates to a tubular insertion device including a tubular device whose flexible portion is inserted into a subject and to an operation support method of supporting an operation of the tubular device.

In a tubular device including a flexible tube portion (insertion portion), such as an endoscope, a device and a method for supporting insertion of the flexible tube portion have been proposed.

For example, U.S. Pat. No. 9,086,340 discloses a tubular insertion device for obtaining operation support information including a plurality of items of external force information regarding external force applied to a flexible tube portion.

### SUMMARY

According to an exemplary embodiment, a tubular insertion device includes a tubular device including a flexible tube portion to be inserted into a subject, a sensor which detects a disposition state of the flexible tube portion in the subject, a prediction calculator which calculates next operation information that is information regarding an operation to be performed next by the tubular device, based on the disposition state detected by the sensor and stored data regarding an operation of the tubular device corresponding to each disposition state of the flexible tube portion, and an output circuit which outputs the next operation information calculated by the prediction calculator.

According to an exemplary embodiment, a tubular insertion device includes a tubular device including a flexible tube portion to be inserted into a subject, a sensor which detects a first disposition state of the flexible tube portion in the subject, a prediction calculator which predicts a second disposition state of the flexible tube portion when a predetermined operation amount is added to the flexible tube portion, based on the first disposition state detected by the sensor, and determines next operation information that is information regarding an operation to be performed next, based on a result of the prediction, and an output portion which outputs the next operation information determined by the prediction calculator.

According to an exemplary embodiment, an operation support method of supporting an operation of an operator of a tubular device of a tubular insertion device, the tubular insertion device including the tubular device including a flexible tube portion to be inserted into a subject, a sensor which detects a disposition state of the flexible tube portion in the subject, and a controller which outputs operation support information based on a result of detection of the sensor, includes, by the controller, calculating next operation information that is information regarding an operation to be performed next by the tubular device, based on the disposition state detected by the sensor and stored data regarding an operation of the tubular device corresponding to each disposition state of the flexible tube portion, and, by the controller, outputting the calculated next operation information as the operation support information.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a diagram schematically showing an example of a tubular insertion device according to a first embodiment of the present invention.

FIG. 2 is an anatomical view schematically showing each part of the large intestine as a subject.

FIG. 3 is a diagram illustrating a configuration of a prediction calculator of the tubular insertion device.

FIG. 4 is a diagram showing a neural network model.

FIG. 5 is a chart showing an example of an insertion assistance control flow according to the first embodiment.

FIG. 6 is a diagram illustrating a plurality of machine learning models in a prediction calculator of a tubular insertion device according to a second embodiment of the present invention.

FIG. 7 is a chart showing an example of an insertion assistance control flow according to the second embodiment.

FIG. 8 is a diagram illustrating the function of an operation validity verification circuit included in the prediction calculator.

FIG. 9 is a diagram illustrating a simulation model in a prediction calculator of a tubular insertion device according to a third embodiment of the present invention.

FIG. 10 is an illustration of an N shape and a shape obtained at the time of loop release.

FIG. 11 is a table showing the input/output relationship when the simulation model of FIG. 9 is constructed by a neural network.

FIG. 12 a chart showing an example of an insertion assistance control flow according to the third embodiment.

### DETAILED DESCRIPTION

Embodiments of the present invention will be described below with reference to the drawings. Hereinafter, an endoscope device including a large-intestine endoscope will be described as an example of a tubular insertion device of the present invention.

### First Embodiment

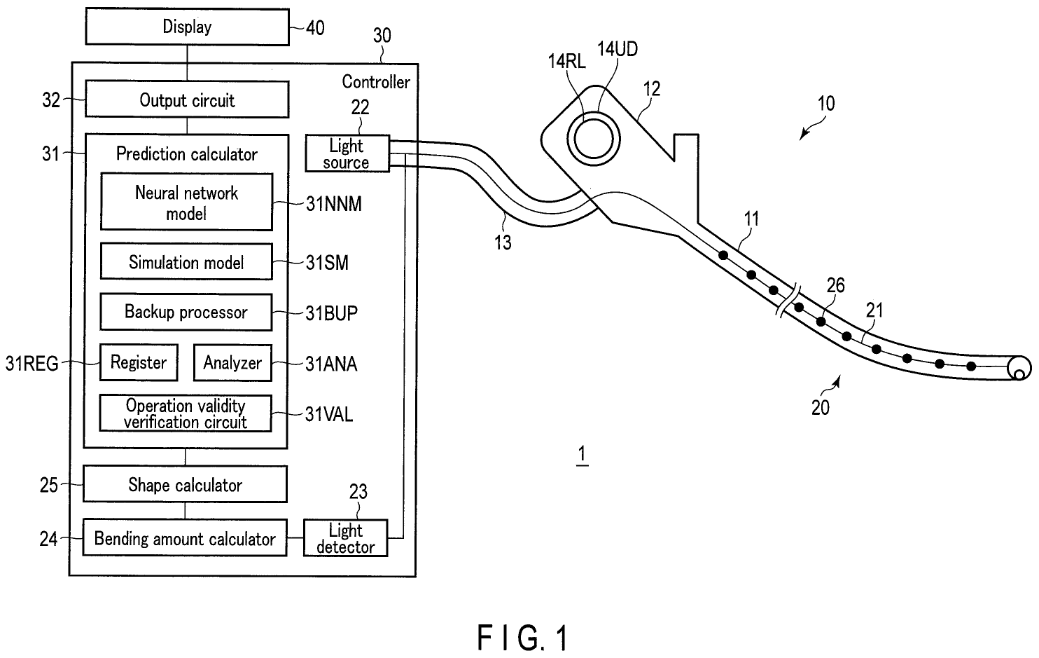

FIG. 1 schematically shows an example of an endoscope device 1. The endoscope device 1 includes a large-intestine endoscope 10, a fiber sensor 20, a controller 30 and a display 40.

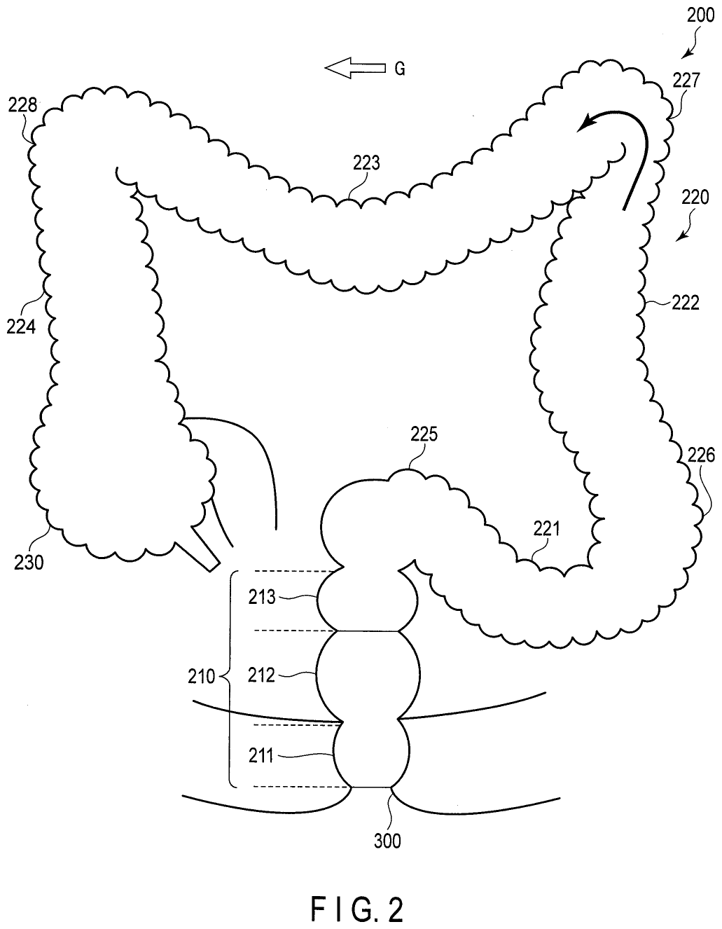

The large-intestine endoscope 10 is a tubular device whose insertion portion 11 is inserted into the large intestine as a subject. First, each part of the large intestine as a subject will be described. FIG. 2 is an anatomical view schematically showing each part of the large intestine 200. The large intestine 200 consists of a rectum 210 connected to an anus 300, a colon 220 connected to the rectum 210, and a cecum 230 connected to the colon 220. The rectum 210 consists of a lower rectum 211, an upper rectum 212 and a rectosigmoid 213 in order from the anal side. The colon 220 consists of a sigmoid colon 221, a descending colon 222, a transverse colon 223 and an ascending colon 224 in order from the rectum 210. The uppermost portion of the sigmoid colon 221 is a sigmoid colon top portion (what is called an S-top) 225. The boundary portion between the sigmoid colon 221 and the descending colon 222 is a sigmoid colon descending colon transition (what is called an SD-Junction (SD-J)) 226. The boundary portion between the descending colon 222 and the transverse colon 223 is a splenic flexure (SF) 227. The boundary portion between the transverse colon 223 and the ascending colon 224 is a hepatic flexure (HF) 228. The S-top 225, SD-J 226, SF 227 and HF 228 are flexing portions of the colon 220. The lower rectum 211 and upper rectum 212 of the rectum 210 and the descending colon 222 and ascending colon 224 of the colon 220 are fixed intestines. On the other hand, the rectosigmoid 213 of the rectum 210, the sigmoid colon 221 and transverse colon 223 of the colon 220, and the cecum 230 are movable intestines. That is, the rectosigmoid 213, sigmoid colon 221, transverse colon 223 and cecum 230 are not fixed but movable in the abdomen.

The large-intestine endoscope 11 includes an operating portion 12 provided on the proximal-end side of the insertion portion 11 and a universal cord 13 for connecting the operating portion 12 and the controller 30, in addition to the insertion portion 11 to be inserted into the large intestine 200.

The insertion portion 11 includes a distal-end rigid portion, an active bending portion provided on the proximal-end side of the distal-end rigid portion, and a passive bending portion provided on the proximal-end side of the bending portion, which are not particularly shown. The distal-end rigid portion includes an illumination optical system including an illumination lens, an observation optical system including an objective lens, an imaging element, and the like, which are not shown. The active bending portion is a flexible portion that is bent by the operation of the operating portion 12, and its bending shape can be changed actively. The passive bending portion is a flexible elongated tubular portion that bends passively.

Since the distal-end rigid portion is a very short portion in the entire length of the insertion portion 11, the “insertion portion 11” will refer to the active bending portion and the passive bending portion below unless otherwise specified. That is, the “insertion portion” is used almost synonymously with a flexible tube portion that can be bent in the tubular device, unless otherwise specified. The “disposition state of the insertion portion 11” detected by the fiber sensor 20 refers to the disposition state of the active and passive bending portions, and the “distal end of the insertion portion 11” is used almost synonymously with the distal end of the active bending portion.

The operating portion 12 is provided with angle knobs 14UD and 14RL used for bending operation of the active bending portion and one or more buttons (not shown) used for various operations including air supply, water supply and suction operations. When an operator operates the angle knob 14UD, the active bending portion bends in a vertical direction with respect to an endoscopic image captured by the imaging device. When the operator operates the angle knob 14RL, the active bending portion bends in a horizontal direction with respect to the endoscopic image. The operating portion 12 is also provided with one or more switches (not shown) to which functions of stopping motion of an endoscopic image, recording an endoscopic image, selecting a focus, etc. according to settings of the controller 30 are assigned.

The fiber sensor 20 is a shape sensor using the loss of amount of light transmission due to bending of an optical fiber 21. The fiber sensor 20 includes a light source 22, a light detector 23, a bending amount calculator 24 and a shape calculator 25, in addition to the optical fiber 21. The light source 22, light detector 23, bending amount calculator 24 and shape calculator 25 are disposed in the interior of the controller 30. Of course, they can be configured separately from the controller 30.

The light source 22 emits light having a plurality of wavelengths. The light source 22 is separate from the light source of an illumination device which emits illumination light for observation and imaging. Note that the illumination device is omitted from FIG. 1.

The optical fiber 21, which guides the light emitted from the light source 22, have flexibility and extend in the interior of the universal cord 13, operating portion 12 and insertion portion 11 from the light source 22. A plurality of detection targets 26 are provided at their corresponding portions of the insertion portion 11 of the optical fibers 21. A plurality of detection targets 26 are arranged at different positions in the longitudinal axis direction of a single optical fiber 21, and a plurality of detection targets 26 are arranged at the same positions or at close positions in the longitudinal axis direction of the single optical fiber 21 and at different positions in the axis circumference direction of the longitudinal axis direction thereof. Alternatively, one detection target 26 may be used for one optical fiber 21. In this case, a plurality of optical fibers 21 are arranged such that the detection target 26 of the optical fiber 21 is disposed at a position different from those of the detection targets 26 of other optical fibers 21 in the longitudinal axis direction of the optical fiber 21. A plurality of optical fibers 21 are also arranged such that a plurality of detection targets 26 are arranged at the same positions or close positions in the longitudinal axis direction of the optical fibers 21 and at different positions in the axis circumference direction of the longitudinal axis direction thereof. If, as described above, a plurality of detection targets 26 are arranged at the same positions or close positions in the longitudinal axis direction of the optical fiber(s) 21 and at different positions in the axis circumference direction of the longitudinal axis direction thereof, the direction of bending as well as the amount of bending can be detected.

That is, the detection target 26 varies the optical characteristics of the optical fiber 21, such as the amount of transmission of light having a predetermined wavelength, in accordance with the amount of bending of the optical fiber. The detection targets 26 differ from each other in the predetermined wavelength. The insertion portion 11 is bent and accordingly the optical fiber 21 is bent. Thus, the amount of light transmission of the optical fiber 21 varies with the amount of bending of the insertion portion 11. A light signal including information regarding the variation of the amount of light transmission is received by the light detector 23. The light detector 23 is configured by, for example, a spectroscope to detect wavelength components of the light signal independently. The light detector 23 may include an element for spectroscopy such as a color filter and a light-receiving element such as a photodiode. The light detector 23 outputs the light signal to the bending amount calculator 24 as state information.

Note that the optical fiber 21 is disposed such that its one end is optically connected to the light source 22, its substantially the middle part in the longitudinal axis direction is folded back at the distal end portion of the insertion portion 11, and its other end is optically connected to the light detector 23. Although not shown, the fiber sensor 20 can be configured such that an optical branching portion is used to optically connect one end of the optical fiber 21 to both the light source 22 and the light detector 23 and the other end of the optical fiber 21, which is located at the distal end portion of the insertion portion 11, is optically connected to a reflecting portion. In this case, the light branching portion guides the light emitted from the light source 22 to the optical fiber 21 and guides the return light reflected by the reflecting portion and guided by the optical fiber 21 to the light detector 23. That is, the light travels through the light source 22, the light branching portion, the optical fiber 21, the reflecting portion, the optical fiber 21, the light branching portion and the light detector 23 in the order described here. The optical branching portion includes, for example, an optical coupler or a half mirror.

The bending amount calculator 24 calculates the amount of bending at the position of each of the detection targets 26 based on the state information from the light detector 23, namely, the change of the light amount corresponding to the bending state of the insertion portion 11 at the position of each of the detection targets 26. The bending amount calculator 24 outputs a result of the calculation to the shape calculator 25.

The shape calculator 25 geometrically converts the amount of bending calculated by the bending amount calculator 24 into a shape to calculate the shape of the insertion portion 11. The shape calculator 25 supplies a prediction calculator 31 with the calculated shape of the insertion portion 11, namely, the disposition state of the insertion portion 11 in the large intestine 200.

The bending amount calculator 24 and the shape calculator 25 may be configured by a processor such as a CPU. In this case, for example, various programs for causing the processor to function as the calculators 24 and 25 are prepared in one of an internal memory and an external memory (neither of which is shown), and the processor executes the programs to perform the functions of the calculators 24 and 25. Alternatively, the calculators 24 and 25 may be configured by a hardware circuit including an application specific integrated circuit (ASIC), a field-programmable gate array (FPGA), and the like.

As described above, the fiber sensor 20 detects a disposition state of the insertion portion 11 in the large intestine 200, namely, a disposition state of the flexible tube portion in a subject, and inputs the detected disposition state of the flexible tube portion to the prediction calculator 31.

The sensor for detecting a disposition state of the insertion portion 11 that is a flexible tube portion in the large intestine 200 as a subject is not limited to the above fiber sensor 20. The sensor may be anything capable of detecting a disposition state of the insertion portion 11. For example, the sensor may be configured by one or a combination of an image sensor for imaging the front and/or side of the distal-end rigid portion of the insertion portion 11, a magnetic position estimation sensor for detecting a spatial position of the insertion portion 11, a bending amount sensor using an optical fiber for detecting the degree of bending of the insertion portion 11 (which can be changed into a shape), a pressure or strain sensor for detecting the degree of contact between the insertion portion 11 and the inner wall of the large intestine 200, a sensor which detects the amount of insertion of the insertion portion 11 into the large intestine 200, a sensor which detects the amount of bending operation to bend the active bending portion of the insertion portion 11, a rotation amount sensor of the insertion portion 11, a gravitational acceleration sensor which detects a direction of the insertion portion 11 with respect to the earth, a working landscape image sensor (which may include an X-ray sensor) which is capable of imaging the insertion portion 11 and a part or the whole of a subject of the insertion portion 11 (including a human body with the large intestine 200 as a subject), and the like.

The controller 30 includes a prediction calculator 31 and an output circuit 32, in addition to the foregoing light source 22, light detector 23, bending amount calculator 24 and shape calculator 25 which constitute a part of the fiber sensor 20 described above. The controller 30 also includes an image processing circuit (not particularly shown) which converts an electrical signal, which is obtained by converting light from a subject by the imaging element of the large-intestine endoscope 10, into a video signal to generate an endoscopic image.

The prediction calculator 31 calculates next operation information, which is information regarding an operation to be performed next by the large-intestine endoscope 10, on the basis of the disposition state of the insertion portion 11 in the large intestine 200 detected by the fiber sensor 20 and the stored data regarding the operation of the large-intestine endoscope 10 according to the disposition state of the insertion portion 11. The prediction calculator 31 is configured by a large-capacity memory and a processor such as a CPU. To increase the processing speed, it is preferable to use a graphics processing unit (GPU) and other dedicated chips.

The output circuit 32 outputs the endoscopic image generated by the image processing circuit (not shown) to the display 40 and causes the display 40 to display the endoscopic image. The output circuit 32 also outputs next operation information, which is a result of calculation of the prediction calculator 31, to the display 40 and causes the display 40 to display it as operation support information. The endoscopic image and the operation support information can be displayed as separate windows on the display screen of the display 40. The display 40 is a general monitor such as a liquid crystal display. A display for displaying the endoscopic image and a display for displaying the operation support information may be independent of each other.

The output circuit 32 can also output a calculation result of the prediction calculator 31 and an endoscopic image to a storage device provided in the controller 30 or placed on a network, and store them therein, which is not particularly shown. Furthermore, the next operation information calculated by the prediction calculator 31 may be output to a sound generator such as a speaker (not shown) and may be output as a guide sound or a warning beep.

Thus, information regarding an operation to be performed next by the large-intestine endoscope 10, which is calculated by the prediction calculator 31, that is, information as to how to operate the large-intestine endoscope 10 next is presented to the operator of the large-intestine endoscope 10 as operation support information. In accordance with the contents presented to the operator, the operator can operate the angle knobs 14UD and 14RL to bend the active bending portion of the insertion portion 11, perform insertion operations of, e.g. pushing, pulling and twisting the insertion portion 11, and perform various operations including air supply, water supply and suction.

The foregoing prediction calculator 31 will be described below in more detail.

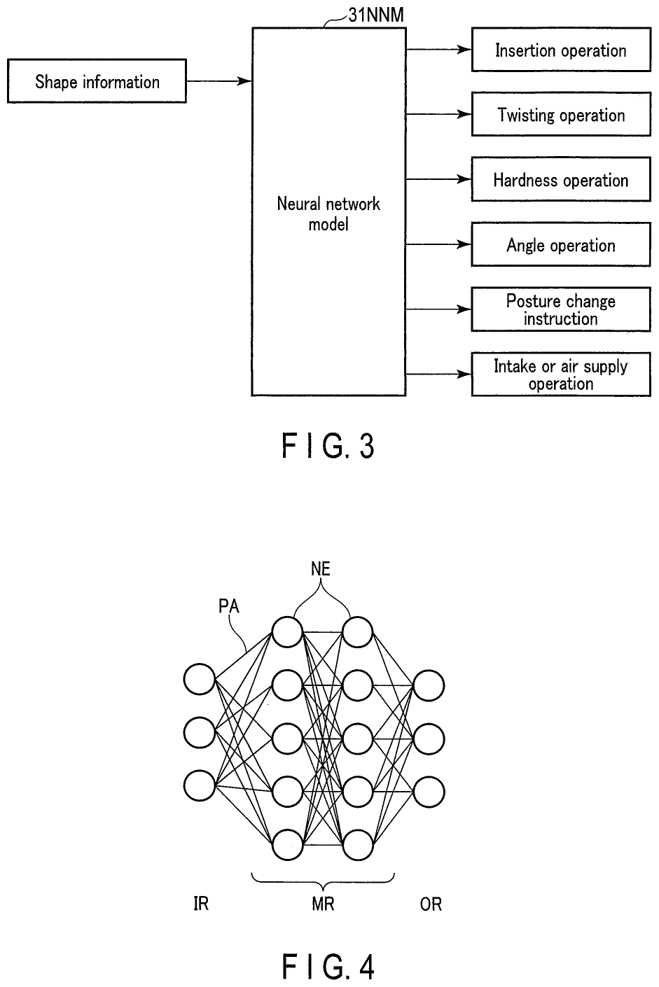

The prediction calculator 31 includes a machine learning model, for example, a neural network model 31NNM based on deep learning of enormous stored data, as shown in FIG. 3. The disposition state of the insertion portion 11 in the large intestine 200 calculated from the amount of bending, i.e., the shape information of the entire insertion portion 11, which is input from the shape calculator 25, is supplied to the neural network model 31NNM.

As shown in FIG. 4, the neural network model includes a plurality of layers including an input layer IR, an intermediate layer MR and an output layer OR. The intermediate layer MR has a multilayer structure. In the neural network model, various parameters PA of the neural network are determined so as to define a relationship between input information in the input layer IR and output information in the output layer OR. The various parameters PA include weighting functions among neurons NE, and are values designed to optimize this function.

In the neural network model 31NNM constituting the prediction calculator 31, the input information in the input layer IR is shape information of the entire insertion portion 11. The output information in the output layer OR is next operation information. That is, the neural network model 31NNM calculates an operation to be performed next by the operator from the input shape information of the entire insertion portion 11. For example, the neural network model 31NNM calculates one or a combination of various operations including “insertion operation” of pushing the insertion portion 11, “twisting operation” of twisting the insertion portion 11, “hardness operation” of changing the hardness of the flexible tube portion when the insertion portion 11 is provided with a hardness changing portion, “angle operation” of changing a bending angle of the active bending portion of the insertion portion by operating the angle knobs 14UD and 14RL, “posture change instruction” to change a posture of the human body having a large intestine 200 that is a subject, and “intake or air supply operation” of performing the intake or air supply.

The neural network model 31NNM described above is constructed from enormous stored data to predict an operation method such that the force exerted on the contact portion of the insertion portion 11 with the large intestine 200 becomes small and that the insertion portion 11 has a target shape. The stored data is constructed on the basis of information obtained from expert's operation information and simulation.

That is, the stored data that constructs the neural network model 31NNM includes input information and output information. The input information is shape information and information regarding shape by expert's operation at time (t). The output information is information of work performed next by the expert. Here, the work performed next is operation information such as a twisting operation of the insertion portion 11, a pushing operation of the insertion portion 11, a hardness changing operation of the insertion portion 11 and a posture change. This operation information is created by determining whether a twisting operation is performed or a pushing operation is performed for the insertion portion 11 from a difference between shape information at time (t) and shape information at next time (t+1). In the neural network model, only information having a difference in shape between time (t) and time (t+1) is learned as teaching data.

Changing the posture when the insertion portion 11 is inserted into the transverse colon 223 as indicated by the arrow in FIG. 2 has the advantage that the insertion direction corresponds to an obtuse angle in relation to gravity G as indicated by an outline arrow in the same figure. Thus, in this time, the posture change is performed. The information regarding this posture change is also obtained as stored data.

In addition, the stored data, namely, the teaching data to be learned includes a result of analysis based on insertion state information, which is information regarding the insertion state of the insertion portion 11 in at least one of inside and outside the large intestine 200. The insertion state information includes at least one of the degree of forward and lateral space of the insertion portion 11, the presence or absence of insertion of the distal end of the insertion portion 11 into the large intestine 200, the degree of the insertion in the direction of a target point in the large intestine 200, the degree of deflection or buckling of the insertion portion 11, the formation of a predetermined loop shape of the insertion portion 11, the size of the predetermined loop shape, and the like.

Furthermore, the teaching data as stored data may include a result of analysis based on a subject information which is information related to the subject itself. The subject information includes at least one of the degree of force applied to the large intestine 200 that is a subject, the degree of pain in the human body having the large intestine 200, the size comparison between the insertion portion 11 and the large intestine 200, the length of the large intestine 200, the characteristic shape of adhesion, diverticulum, etc., the history of surgery, a surgical scar, the heart rate of the human body, the movement of the human body, perforation information, the endoscope trouble, the treatment tool trouble, and the like.

Furthermore, for example, the external force which is force exerted on a contact portion of the flexible tube portion of the insertion portion 11 until the predetermined loop shape of the insertion portion 11 is released is great. It is desirable that the teaching data as stored data is not learned when the external force is great. Note that information of the external force may be calculated from the shape of the insertion portion 11 or calculated by simulation. In the case of calculation by simulation, an optimum release method in which the above external force clarified by simulation becomes small, such as the finite element method (FEM) and the mechanism analysis, may be learned. The external force is force acting near the S-top 225 and force acting on the transverse colon 223. The method of releasing the loop shape is an operation method clarified by an optimization operation (local or global optimization technique). The neural network model 31NNM thus learned is constructed as shown in FIG. 4.

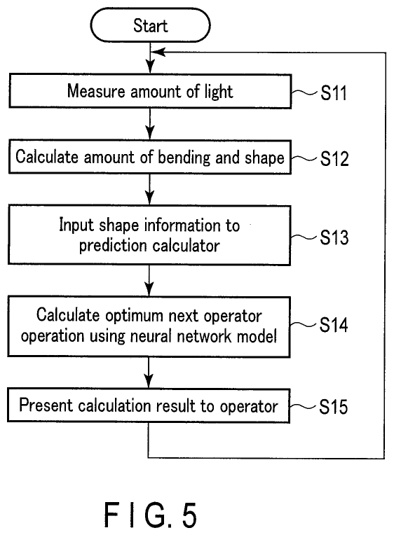

The insertion support control operation in the endoscope device 1 including the prediction calculator 31 having the neural network model 31NNM will be described with reference to FIG. 5.

First, the light source 22 of the fiber sensor 20 causes light to enter the optical fiber 21, and the light detector 23 measures the amount of light whose transmission amount is changed according to the bending state by each of the detection targets 26 provided in the optical fiber 21 (step S11). Then, the bending amount calculator 24 of the fiber sensor 20 calculates the amount of bending in each of the detection targets 26 from a change in the amount of light measured by the light detector 23, and the shape calculator 25 calculates the shape of the optical fiber 21, that is, the shape of the insertion portion 11, based on the amount of bending in each of the detection targets 26 calculated by the bending amount calculator 24 (step S12). The shape calculator 25 input shape information indicating the calculated shape of the insertion section 11 to the prediction calculator 31 (step S13).

The prediction calculator 31 calculates next operation information as the optimum next operator operation using the neural network model 31NNM (step S14). Then, the prediction calculator 31 outputs the next operation information, which is a calculation result, to the output circuit 32, and the output circuit 32 displays it on the display 40 to present operation support information indicating what operation should be performed next to the operator (step S15). After that, the process is repeated from step S11 described above.

By repeatedly executing the routine of steps S11 to S15 as described above, for example, when a loop shape is generated in the insertion portion 11 and a desired shape for releasing the loop shape is a linear shape, the prediction calculator 31 can calculate the following next operation information and present the operation support information. That is, an insertion operation of “pushing” is first presented to the operator, and then, when the insertion portion 11 starts to bend into a cylindrical shape, the hardness operation of “changing hardness,” the posture change instruction to “change a posture,” and the like are provided. When the insertion portion 11 has a desired shape for releasing the loop, the twisting operation of “twisting to the right” or “twisting to the left” and the insertion operation of “pushing” or “pulling” are provided for the operator.

In executing the above routine of steps S11 to S15, when an operator provides an end instruction by, for example, operating an input switch (not shown) provided in or connected to the controller 30, the routine is ended.

As described above, the endoscope device 1 as a tubular insertion device according to the first embodiment includes a large-intestine endoscope 10 that is a tubular device in which an insertion portion 11 as a flexible tube portion is inserted into a large intestine 200 that is a subject, a fiber sensor 20 that is a sensor which detects a disposition state of the insertion portion 11 in the large intestine 200, for example, shape information of the insertion portion 11, a prediction calculator 31 including a neural network model 31NNM that is a machine learning model which calculates next operation information that is information regarding an operation to be performed next by the large-intestine endoscope 10, based on the shape information of the insertion portion 11 detected by the fiber sensor 20 and stored data associated with an operation of the large-intestine endoscope 10 corresponding to each shape information of the insertion portion 11, and an output circuit 32 which outputs the next operation information calculated by the prediction calculator 31.

Therefore, according to the endoscope device 1 as the tubular insertion device according to the first embodiment, the optimum operation support information can be obtained from the current shape of the insertion portion 11 and thus accurate operation information corresponding to the insertion state can be output as operation support information.

For example, it is difficult to master a technique of inserting the endoscope into a portion of the sigmoid colon 221 in large intestine endoscopy. It is particularly difficult for an inexperienced operator to insert the distal end of the insertion portion 11 into a lumen subsequent to a bending portion of a moving intestine. According to the present embodiment, however, an operator's operation can be simplified by providing appropriate insertion support by the endoscope device 1. Therefore, even an operator with little experience can perform an operation similar to that of an experienced operator.

Note that the input information is shape information in the neural network model 31NNM shown in FIG. 3. As the input information, the shape information of the insertion portion 11 may be input as it is, or a characteristic shape calculated from the shape information may be input. The characteristic shape includes, for example, the number of flexion points (where the bending direction is reversed) of the insertion portion 11, the magnitude of curvature at each of the flexion points, and the like. In addition, as information other than the shape information, image information, hardness change information, angle operation amount, and the like may be input.

### Second Embodiment

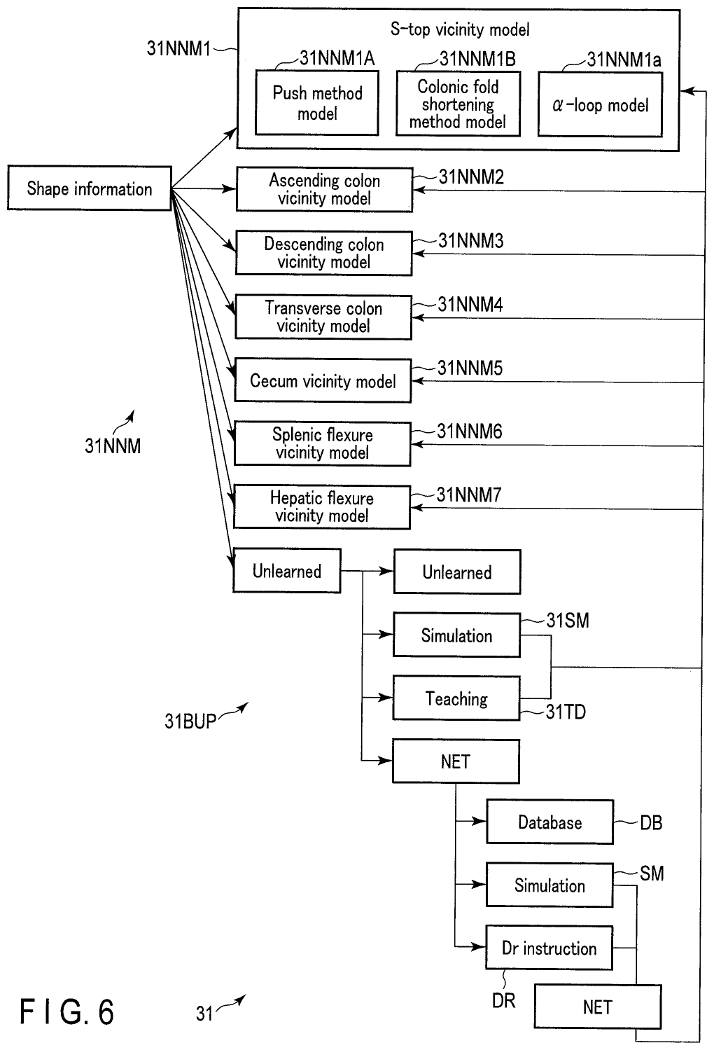

A second embodiment of the present invention will be described with reference to FIGS. 6 to 8. In the following, portions different from those of the first embodiment will chiefly be described, and components like those of the first embodiment will be denoted by reference symbols like those of the first embodiment and their descriptions will be omitted.

The first embodiment is directed to a model to simulate all operations of a single neural network model 31NNM; however, in order to obtain the optimum operation support information for the entire large intestine 200 by the single model, an enormous amount of data learning is required. In the second embodiment, therefore, the neural network model 31NNM is constructed as a plurality of neural network models corresponding to portions of the large intestine 200 shown in FIG. 2. For example, as shown in FIG. 6, the prediction calculator 31 includes an S-top vicinity model 31NNM1 corresponding to the vicinity of the S-top 225, an ascending colon vicinity model 31NNM2 corresponding to the vicinity of the ascending colon 224, a descending colon vicinity model 31NNM3 corresponding to the vicinity of the descending colon 222, a transverse colon vicinity model 31NNM4 corresponding to the vicinity of the transverse colon 223, a cecum vicinity model 31NNM5 corresponding to the vicinity of the cecum 230, a splenic flexure vicinity model 31NNM6 corresponding to the vicinity of the SF 227, a hepatic flexure vicinity model 31NNM7 corresponding to the vicinity of the HF 228, and the like.

In order to prevent an operator from performing an inappropriate operation when unlearned data, i.e., first shape information is input from the shape calculator 25, the prediction calculator 31 may include a simulator or the like, which calculates, in real time, alternative information regarding an operation to be performed next by a simulation model 31SM. For example, the prediction calculator 31 uses a simulation model to calculate an amount of force acting on a portion of contact between the insertion portion 11 inserted into the large intestine 200 and the large intestine 200 and calculate a release method for reducing the amount of force as alternative information.

The determination as to whether unlearned data is input can be made according to, for example, whether the degree of matching between the shape information of the insertion portion 11 detected by the fiber sensor 20 and the stored data of the neural network model 31NNM is low, that is, whether it greatly differs from the learned teaching data. Alternatively, a neural network model in which various shapes and NG shapes are learned in advance may be provided. In this case, learning has only to be made by intentionally creating several patterns of a shape or the like which clearly differs from the shape learned by the neural network model 31NNM for the next operation information calculation.

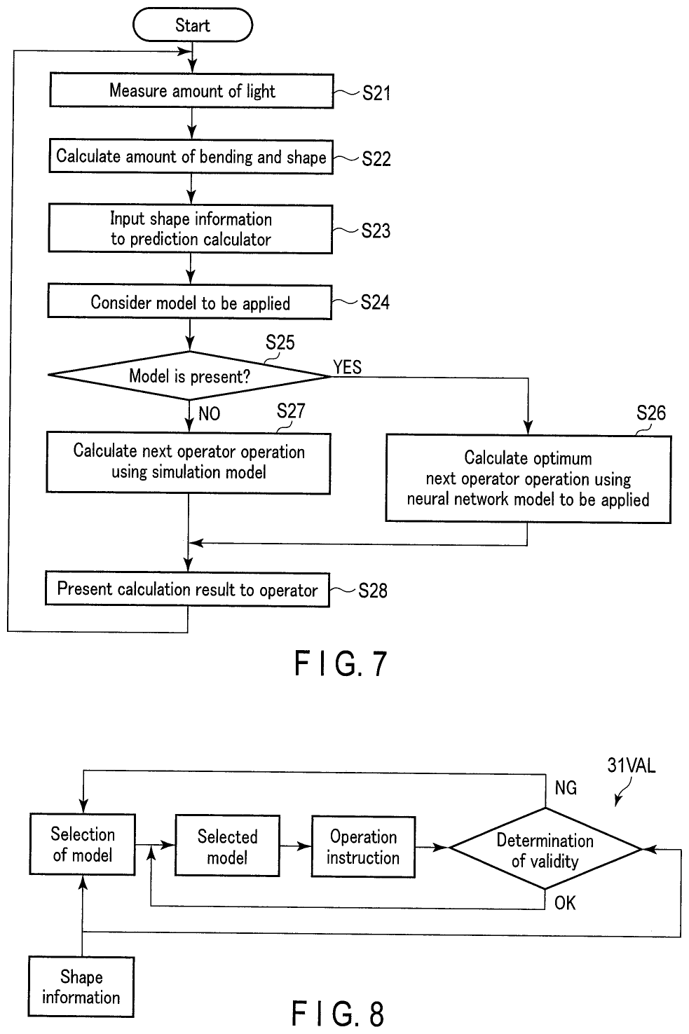

The insertion support control operation in the endoscope device 1 of the present embodiment will now be described with reference to FIG. 7. Note that steps S21 to S23 are the same as steps S11 to S13 in the first embodiment and thus their descriptions will be omitted.

The prediction calculator 31 considers which neural network model is applied based on the input shape information of the insertion portion 11 (step S24). Then, if a neural network model to be applied is present (Yes in step S25), the prediction calculator 31 selects the neural network model, inputs the shape information of the insertion portion 11 to the neural network model, and calculates next operation information as the optimum next operator operation according to the neural network model (step S26).

If there is no neural network model to be applied (No in step S25), the prediction calculator 31 uses the simulation model 31SM to calculate alternative operation information which is not optimal or dangerous but is the next operator operation (step S27). The alternative operation information so obtained may be registered as new stored data, or teaching data in the neural network model corresponding to the shape information of the insertion portion 11.

If the next operation information or the alternative operation information is calculated as described above, the prediction calculator 31 outputs the next operation information or the alternative operation information to the output circuit 32 as a result of the calculation, and the output circuit 32 displays it on the display 40 to present the operator with operation support information indicating what operation should be performed next (step S28). After that, the process is repeated from above step S21.

The above routine of steps S21 to S28 is ended when an operator provides an end instruction by, for example, operating an input switch (not shown) provided in or connected to the controller 30.

As described above, according to the endoscope device 1 as the tubular insertion device according to the second embodiment, the prediction calculator 31 includes a plurality of neural network models (31NNM1 to 31NNM7) to be selected according to the disposition state of the insertion portion 11, for example, the shape information thereof to select a neural network model for use in calculating next operation information in accordance with the shape information of the insertion portion 11 in the large intestine 200 detected by the fiber sensor 20.

Using a neural network model constructed with patterns that can be formed to limit a portion, such as the S-top 225 as described above, makes it possible to calculate more optimum next operation information and decrease the probability of issuing an erroneous operation instruction. It is also possible to present optimum next operation information suitable for various operator operations. Since, furthermore, the neural network model is suitable for each part, a high-accuracy neural network model can be constructed with a small amount of stored data for a single model.

Note that the neural network model corresponding to each part of the large intestine 200 may be further subdivided to use a neural network model corresponding to an operator's operation technique. For example, the S-top vicinity model 31NNM1 includes a push method model 31NNM1A corresponding to the push method known as one of the insertion techniques, a colonic fold shortening method model 31NNM1B corresponding to the insertion technique by shortening the colonic fold through bending, which is known as one of the insertion techniques, and the like. If it is determined from the shape information of the insertion portion 11 that the operator aims at the release of a loop using the push method in the vicinity of the S-top 225, the push method model 31NNM1A is used to calculate next operation information. If it is determined that the operator aims at the colonic fold shortening method, the colonic fold shortening method model 31NNM1B is used to calculate next operation information. In addition, the neural network model may be constructed according to the shape of a loop generated in the insertion portion 11. For example, the S-top vicinity model 31NNM1 includes an α-loop model 31NNM1a corresponding to the shape of an α-loop, and the like. If it is determined from the shape information of the insertion portion 11 that an α loop is generated, the α-loop model 31NNM1a is utilized to calculate next operation information.

According to the present embodiment, the prediction calculator 31 includes, for example, a simulator as a backup processor 31BUP that acquires alternative information regarding an operation to be performed next by the large-intestine endoscope 10 by a calculation method (simulation model) other than the next-operation information calculation method (neural network model) when the degree of matching between the shape information of the insertion portion 11 detected by the fiber sensor 20 and the stored data is low (a difference between the shape information and the learned teaching data is great). Accordingly, even though unlearned shape information is input, an operator can be prevented from performing an inappropriate operation, especially a dangerous operation.

As shown in FIG. 1, according to the present embodiment, the prediction calculator 31 may include a register 31REG which increases the stored data based on the shape information of the insertion portion 11 detected by the fiber sensor 20 and the alternative information. Therefore, when unlearned data is input, the alternative operation information calculated by the backup processor 31BUP can be used as new learning data.

The backup processor 31BUP may be configured such that an expert operator or the like performs an unlearned operation to create teaching data 31TD newly. The teaching data 31TD may be presented as alternative operation information to an operator having little experience, or may be registered as new learning data by the register 31REG. This registration may be made in accordance with the level of the operator. For example, the registration according to the level of the operator may be made as a low-level release method when the external force acting on the contact portion of the insertion portion 11 at the time of releasing the loop shape is small and made as a high-level release method when the external force is small.

Alternatively, the backup processor 31BUP may be designed not to support an operation in order to prevent the operator from performing an inappropriate operation. That is, when the degree of matching between the shape information of the insertion portion 11 detected by the fiber sensor 20 and the stored data is low (a difference between the shape information and the learned teaching data is large), the backup processor 31BUP outputs a result of incapability of calculation to the output circuit 32. Upon receiving the result of incapability of calculation, the output circuit 32 does not output the result of incapability of calculation to the display 40 or it causes the display 40 to display that the next operation information cannot be output.

If the prediction calculator 31 has a communication function, the backup processor 31BUP may cause a high-performance computer or the like to perform a loop release operation calculation at high speed through a network NET. That is, the backup processor 31BUP requests a server to calculate alternative information using the simulation model SM, through the network NET, and receives the alternative information, which is a result of the calculation, from the server through the network NET. In this case, too, the received alternative information is not only presented to the operator through the output circuit 32, but also may be registered as new learning data by the register 31REG.

Alternatively, the backup processor 31BUP may transmit information such as shape information to another operator, for example, a doctor (Dr.) skilled in operation in real time through the network NET, and receive feedback (Dr instruction DR) from the another operator. That is, the backup processor 31BUP requests an input device to transmit alternative information through the network NET and receives the alternative information from the input device through the network NET. In this case, too, the received alternative information is not only presented to the operator through the output circuit 32, but also may be registered as new learning data by the register 31REG.

In addition to the above, the backup processor 31BUP may transmit, through the network NET, unlearned data to a provider who provides a neural network model and store the unlearned data in the database DB of the provider so that the unlearned data can be used for teaching data for a neural network model provided next by the provider.

The operator may select one of the foregoing operations to be performed by the backup processor 31BUP by operating an input switch (not shown) provided in or connected to the controller 30, for example.

The classification into each neural network model may be performed not based on the deep learning by the shape information, but based on other machine learning, for example, an algorithm such as a bag of words. The model classification may also be performed based on time series data. In the case of the large intestine 200, the insertion portion 11 passes through the S-top 225 and then the sigmoid colon 221 and the descending colon 222 to reach the SF 227 and thus the SF 227 does not suddenly appear after the S-top 225. Therefore, the classification may be performed in accordance with the amount of insertion, the time-series order, and the like. This configuration makes it possible to provide the operator with the optimum operation support with high accuracy.

The next operation information and the alternative operation information to be presented may be different from each other depending on the level of an operator who operates the large-intestine endoscope 10, including whether they are presented or not. The operator level is preferably switched by operating the input switch (not shown) provided in or connected to the controller 30, for example.

In addition, as shown in FIG. 1, the prediction calculator 31 may include an operation validity verification circuit 31VAL which determines whether an operation is correctly performed in accordance with the calculated next operation information. The operation validity verification circuit 31VAL includes a feedback path which selects another neural network model to calculate next operation information when a result of the determination is negative. The feedback path may include an instruction to stop the operation or an instruction to decrease the speed of the operation.

For example, the prediction calculator 31 selects a neural network model based on the input shape information of the insertion portion 11, calculates next operation information using the selected model, and presents the display 40 with an instruction to perform an operation next as support information through the output circuit 32. Accordingly, the operator performs the operation. As shown in FIG. 8, the operation validity verification circuit 31VAL compares and verifies the shape information corresponding to the next operation information, which will be obtained by this operator operation, and the actual shape information of the insertion portion 11 in the large intestine 200 detected by the fiber sensor 20. If they are different, the operation validity verification circuit 31VAL selects another neural network model. Furthermore, when there is, for example, adhesion, even if the insertion portion 11 is operated as instructed, a phenomenon that the insertion portion 11 does not move occurs. The operation validity verification circuit 31VAL calculates an amount of force exerted on the outside by the insertion portion 11 from the shape information to determine that the operation is not correctly performed when the amount of force is too large and to update the selection of the neural network model so as to instruct it to perform an operation suitable for the case.

As described above, even if the operator does not operate as instructed or even if the insertion state differs from the expected state, feedback can be quickly applied.

As shown in FIG. 1, the prediction calculator 31 may also include an analyzer 31ANA which analyzes the operator's operation of the large-intestine endoscope 10. The analyzer 31ANA conducts an analysis of leveling, stored data classification and the like, based on a high degree of insertion condition of the insertion portion 11 in the large intestine 200 as a subject in the direction of a target point. The high degree of insertion condition includes whether the of the insertion portion 11 is smooth, whether the insertion speed of the insertion portion 11 is appropriate, whether the arrival time to the target point of the insertion portion 11 is appropriate, whether there is an oversight, whether the insertion portion 11 does not form a loop, whether the loop formation is small, whether the load on a subject is small, whether an accidental symptom occurs by image determination, whether the degree of progress inhibition in front of the screen is low, whether a lumen is caught in the center of an image, whether the insertion portion 11 is largely moved, and the like. When a plurality of items of next operation information are calculated for certain shape information, the leveling makes it possible to preferentially present next operation information corresponding to a high-level operator operation. Alternatively, it can be used to contribute to a burden on patients of a hospital or the like and the improvement of the quality of medical care if operators are placed on the optimum positions in accordance with the difficulty in insertion into the patients and a high-quality operation is extracted and shared.

The register 31REG may increase the stored data based on the result of the analyzer 31ANA. Thus, a skilled operator who does not need much next operation information to be presented is caused to stop the function of calculating the next operation information and to play a role of increasing the stored data, thereby improving the performance of the entire system.

The backup processor 31BUP calculates the alternative operation information with unlearned data input when the shape information of the insertion portion 11 detected by the fiber sensor 20 greatly differs from the learned teaching data. The backup processor 31BUP may calculate alternative information when it is confirmed that the chronological continuity of the next operation information is broken. For example, it is the case where during an operation for releasing the loop shape, an operation other than the release is obtained as next operation information.

### Third Embodiment

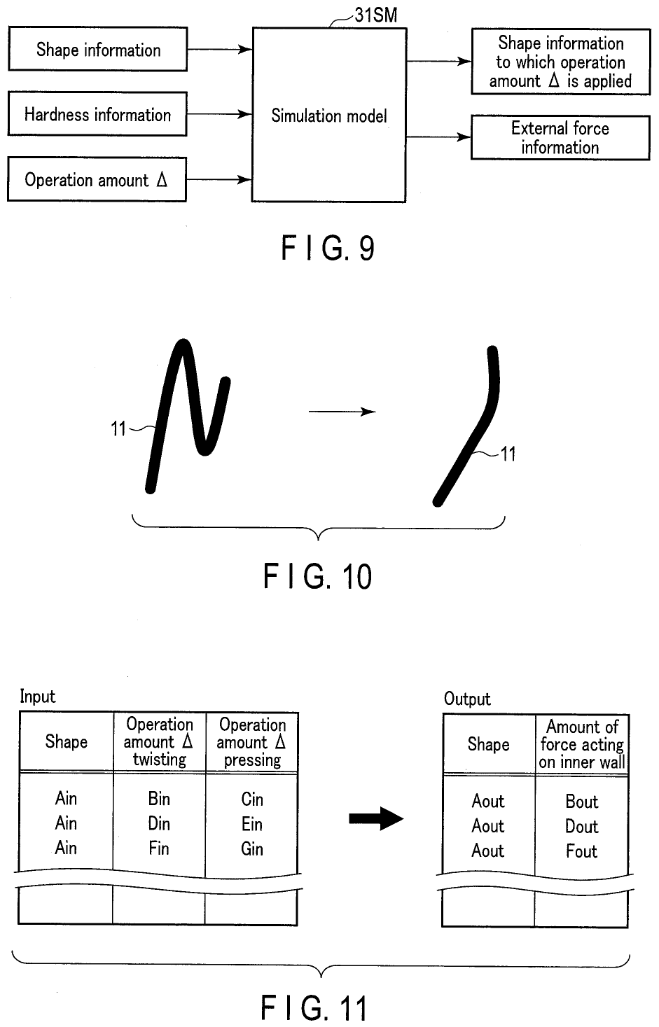

A third embodiment of the present invention will be described with reference to FIGS. 9 to 12. In the following, portions different from those of the first embodiment will chiefly be described, and components like those of the first embodiment will be denoted by reference symbols like those of the first embodiment and their descriptions will be omitted.

In the first embodiment, the prediction calculator 31 includes a machine learning model such as the neural network model 31NNM, whereas a prediction calculator 31 of the third embodiment 3 includes a simulation model 31SM instead, as shown in FIG. 9. In this case, it may be clarified from the simulation model 31SM how external force information and shape information change when operation amount A is added, and the optimum operation method may be presented as operation support information. The operation amount is equal to an insertion amount of the insertion portion 11, a twisting amount, and the like. The operation amount can include hardness information when the insertion portion 11 includes a hardness changing portion. The reason for using the hardness information as the operation amount is that when the value is changed, the rigidity value of the simulation model 31SM is changed and an influence is exerted on the shape change and the like.

For example, when the current shape of the insertion portion 11 is a loop shape, a stack shape and the like, the prediction calculator 31 causes the simulation model 31SM to analyze what operation method should be executed in order to form the insertion portion 11 to have a desired shape for releasing the current shape. The desired shape means, for example, that the insertion portion 11 having an N shape is formed into a straight shape as shown in FIG. 10. The loop shape releasing method is an operation method that is clarified by the optimization calculation (local optimization method and global optimization method).

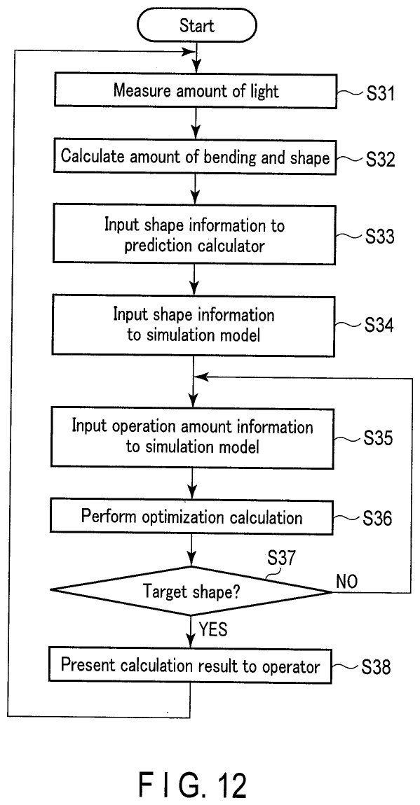

An insertion support control operation in the endoscope device 1 of the present embodiment will be described with reference to FIG. 12. Note that steps S31 to S33 are the same as steps S11 to S13 in the first embodiment 1 and thus their descriptions will be omitted.

The prediction calculator 31 inputs the input shape information of the insertion portion 11 to the simulation model 31SM (step S34). The prediction calculator 31 also inputs operation amount information to the simulation model 31SM (step S35). Then, the simulation model 31SM performs an optimization calculation based on the input shape information and the operation amount information (step S36). If a target shape or a desired shape is not obtained by the optimization calculation (No in step S37), the process returns to step S35 described above, and the prediction calculator 31 inputs other operation amount information to the simulation model 31SM.

If a target shape or a desired shape is obtained by the optimization calculation by repeating the routine of above steps S35 to S37 (Yes in step S37), the prediction calculator 31 outputs the operation amount information at the time to the output circuit 32 as next operation information that is a result of the calculation, and the output circuit 32 displays it on the display 40, thus presenting the operator with operation support information indicating what operation should be performed next (step S38). After that, the process is repeated from step S31.

The routine of above steps S31 to S38 is terminated when an operator provides an end instruction by, for example, operating an input switch (not shown) provided in or connected to the controller 30.

Since the simulation model 31SM needs to be analyzed in real time, high-speed calculation can be performed if a simple simulation model in which an input/output relation is converted into an approximate expression is used.

On the other hand, real-time calculation cannot be performed in the finite element method or the mechanism analysis, which is a detailed simulation. As the simulation model 31SM, therefore, a neural network model may be used in which the shape information and each operation amount A (and hardness information in some cases) are input values as input information and the shape after the operation amount A is input and the information of force of the insertion portion 11 applied when the insertion portion 11 is in contact with the inner wall of the lumen are output values, as shown in FIG. 11. These relations are created as the neural network model from input/output information of enormous simulation.

Since the created neural network model becomes a simulator capable of high-precision and high-speed operation, real-time convergence calculation can be performed, and an operator can be provided with a releasing method in which the force acting on the contact portion of the insertion portion 11 is reduced.

As described above, according to the endoscope device 1 as the tubular insertion device according to the third embodiment, the prediction calculator 31 can calculate next operation information by the simulation model 31SM and present it as operation support information.

In the tubular insertion device according to the third embodiment, too, the simulation model 31SM may be subdivided according to each part of a subject, like in the second embodiment.

Each of the embodiments of the present invention has been so far described with reference to the endoscope device 1 including the large-intestine endoscope 10. The tubular insertion device of the present invention is not limited to the endoscope device, but may be any tubular device if it includes a flexible tube portion. For example, it may be a medical endoscope for a body cavity other than the large intestine 200 as a subject and an industrial endoscope for a tube cavity such as a pipe and an engine.

In addition, the present invention is not limited to a device in which an operator performs an operation of inserting a flexible tube portion, but can be applied to a robot technology in which a flexible tube portion is automatically inserted into a subject. In this case, the output circuit 32 supplies information regarding an operation which is calculated by the prediction calculator 31 and which is to be performed next to not only the display 40 but also a robot controller, with the result that an automatic operation based on the information can be performed. Thus, an operator who operates the tubular device is not limited to human beings, but may be a machine.

Additional advantages and modifications will readily occur to those skilled in the art. Therefore, the invention in its broader aspects is not limited to the specific details, representative devices, and illustrated examples shown and described herein. Accordingly, various modifications may be made without departing from the spirit or scope of the general inventive concept as defined by the appended claims and their equivalents.

What is claimed is:

1. A tubular insertion device comprising:
a tubular device including a flexible tube portion to be inserted into a subject;
a sensor which detects a disposition state of the flexible tube portion in the subject;
a prediction calculator which calculates next operation information that is information regarding an operation to be performed next by the tubular device, based on the disposition state detected by the sensor and stored data regarding an operation of the tubular device corresponding to each disposition state of the flexible tube portion; and
an output circuit which outputs the next operation information calculated by the prediction calculator.

2. The tubular insertion device according to claim 1, wherein the stored data is a result of analysis based on insertion state information of the flexible tube portion, the insertion state information being information regarding an insertion state of the flexible tube portion at least one of inside and outside the subject.

3. The tubular insertion device according to claim 1, wherein the stored data is a result of analysis based on subject information that is information regarding the subject.

4. The tubular insertion device according to claim 1, wherein the prediction calculator includes at least one of a machine learning model based on the stored data and a simulation model based on the stored data.

5. The tubular insertion device according to claim 4, wherein the at least one of the machine learning model and the simulation model is constructed from enormous stored data regarding an operation of the tubular device.

6. The tubular insertion device according to claim 1, wherein the next operation information calculated by the prediction calculator includes at least one of: information of a twisting direction of the flexible tube portion; information of a bending direction of an active bending portion provided at the flexible tube portion; information of an amount of insertion of the flexible tube portion into the subject; information of hardness of the flexible tube portion to be changed by a hardness changing portion provided at the flexible tube portion; and information of an instruction to change a posture of the subject.

7. The tubular insertion device according to claim 1, wherein the prediction calculator includes a plurality of items of the stored data selected by the disposition state of the flexible tube portion, and selects the stored data to be used to calculate the next operation information in accordance with the disposition state of the flexible tube portion in the subject detected by the sensor.

8. The tubular insertion device according to claim 1, wherein the prediction calculator includes an operation validity verification circuit which determines whether an operation is correctly performed in accordance with the calculated next operation information.

9. The tubular insertion device according to claim 8, wherein:
the prediction calculator includes at least one of: a plurality of machine learning models based on the stored data; and a plurality of simulation models based on the stored data; and
the operation validity verification circuit includes a feedback path which selects another model to cause the prediction calculator to calculate the next operation information when a result of the determination is negative.

10. The tubular insertion device according to claim 8, wherein the operation validity verification circuit has a function of: comparing a disposition state of the flexible tube portion corresponding to the next operation information output from the output circuit and an actual disposition state of the flexible tube portion in the subject detected by the sensor; and verifying whether the disposition state and the actual disposition state differ from each other.

11. The tubular insertion device according to claim 1, wherein:
the prediction calculator outputs a result of incapability of calculation to the output circuit when a degree of matching between the disposition state detected by the sensor and the stored data is low; and
when the output circuit receives the result of incapability of calculation from the prediction calculator, the output circuit performs one of: inhibiting from outputting the result of incapability of calculation; and outputting a fact that the next operation information calculated by the prediction calculator cannot be output.

12. The tubular insertion device according to claim 1, wherein the prediction calculator includes a backup processor which acquires alternative information regarding an operation to be performed next by the tubular device by a calculation method other than a next operation information calculation method when a degree of matching between the disposition state detected by the sensor and the stored data is low.

13. The tubular insertion device according to claim 12, wherein the backup processor requests an input device, which inputs the alternative information, to transmit the alternative information through a network, and receives the alternative information from the input device through the network.

14. The tubular insertion device according to claim 1, wherein the prediction calculator selects the next operation information in accordance with a level of an operator who operates the tubular device.

15. The tubular insertion device according to claim 4, wherein the simulation model is converted into one of a machine learning model and an approximate expression from an input/output relation.

16. The tubular insertion device according to claim 1, wherein the prediction calculator further includes an analyzer which analyzes an operation of the tubular device operated by an operator.

17. The tubular insertion device according to claim 16, wherein the prediction calculator further includes a register which increases the stored data based on a result of the analyzer.

18. The tubular insertion device according to claim 1, wherein the tubular device includes an endoscope.

19. A tubular insertion device comprising:
a tubular device including a flexible tube portion to be inserted into a subject;
a sensor which detects a first disposition state of the flexible tube portion in the subject;
a prediction calculator which predicts a second disposition state of the flexible tube portion when a predetermined operation amount is added to the flexible tube portion, based on the first disposition state detected by the sensor, and determines next operation information that is information regarding an operation to be performed next, based on a result of the prediction; and
an output portion which outputs the next operation information determined by the prediction calculator.

20. An operation support method of supporting an operation of an operator of a tubular device of a tubular insertion device, the tubular insertion device including the tubular device including a flexible tube portion to be inserted into a subject, a sensor which detects a disposition state of the flexible tube portion in the subject, and a controller which outputs operation support information based on a result of detection of the sensor, the method comprising:
by the controller, calculating next operation information that is information regarding an operation to be performed next by the tubular device, based on the disposition state detected by the sensor and stored data regarding an operation of the tubular device corresponding to each disposition state of the flexible tube portion; and
by the controller, outputting the calculated next operation information as the operation support information.
